(19) United States
(12) Patent Application Publication
(10) Pub. No.: US 2021/0011013 A1
(43) Pub. Date: Jan. 14, 2021

(54) Novel Biochip Substrate, Preparation Method and Application Thereof

(71) Applicant: DALIAN UNIVERSITY OF TECHNOLOGY, Dalian, Liaoning (CN)

(72) Inventors: Fang CHENG, Dalian, Liaoning (CN); Jicheng DONG, Dalian, Liaoning (CN); Bingbing SUN, Dalian, Liaoning (CN); Huanan WANG, Dalian, Liaoning (CN)

(21) Appl. No.: 16/980,724

(22) PCT Filed: May 18, 2018
(86) PCT No.: PCT/CN2018/087454

§ 371 Date: Sep. 14, 2020

(30) Foreign Application Priority Data
Apr. 4, 2018 (CN) 201810301488.X

(51) Int. Cl. G01N 33/543; C12Q 1/6837; C07C 317/18; C07C 315/04; G01N 21/64

(52) U.S. Cl. CPC G01N 33/54353; C12Q 1/6837; G01N 2021/6439; C07C 315/04; G01N 21/6428; C07C 317/18

(57) ABSTRACT

The present disclosure discloses a novel biochip substrate, a preparation method and an application thereof. The surface of the novel biochip substrate contains active vinyl sulfone groups. The preparation method involves a one-step reaction of a compound containing vinyl sulfone groups on both ends with a silicon-hydroxyl group on the surface of a silicon-based biochip substrate material under catalytic conditions, to prepare the biochip substrate. The application immobilizes biomacromolecules by conducting Michael addition of amino or sulfydryl group in biomacromolecules and the vinyl sulfone group on the surface of the biochip substrate, realizing biological functionalization thereof. The biochip substrate has high-density active vinyl sulfone groups, which can be used for immobilization of various biomolecules with mild fixation conditions and simple operation. The preparation method does not require complex pretreatment processes, and has high operability and reproducibility, low cost, mild reaction conditions, simple operation, and environmentally friendly, which is a broad-spectrum biochip substrate with great potential.

### TECHNICAL FIELD

The present disclosure belongs to the technical field of biochip, and in particular relates to a novel biochip substrate and a preparation method thereof, also to the application of the substrate.

### BACKGROUND ART

Biochip technology is a highly innovative technology, covering research fields of biology, chemistry, medicine, physics, material science, electronic technique, bioinformatics, confidential instrument, etc. Biochip refers to the supports (silicon wafer, glass slide or high-molecular polymer slice) immobilized and arranged with labeled bioprobes, after specific affinity reaction occurs between the samples to be detected and the probes on the supports, the labeled signals on each probe are scanned and analyzed by computer software, so as to complete the detection of biological substances such as DNA, RNA, polypeptide and protein. Biochip is the basis of micro biochemical analysis, compared with traditional analysis methods, it has significant advantages: various analytes can be researched on the same sample at the same time, small sample sizes required, low consumption of rare reagents, quantification at a highly micro-level, and high throughput.

For silicon materials, such as silicon wafer, glass, optical fiber and other common biochip underlays, there are a variety of biochip substrate preparation methods at present. Organic silane coupling agent is a common chemical reagent for surface treatment, which is used in amino substrate, aldehyde substrate and epoxy substrate that commonly used in biochips. During applications, aldehyde substrate immobilizes biomacromolecules through Schiff base formed by the aldehyde groups on its surface and the amino groups in biomolecules. After immobilization, appropriate blocking agents are needed to block the unreacted aldehyde group on the substrate to reduce nonspecific adsorption. Amino substrate immobilizes a large number of DNA molecules on its surface by electrostatic interaction between positively charged primary amine groups on the surface under neutral conditions and negatively charged phosphate groups in DNA molecules. Ultraviolet irradiation or heating of the chip can further lead to the formation of covalent bonds between DNA molecules and amino substrates. The mechanism of immobilizing biomolecules on epoxy substrate is through nucleophilic opening of the epoxy ring by amino groups on biomolecules. Silane coupling agents covalently tether organic molecules onto the material surface by Si—O—Si bonds. However, silane coupling agent is sensitive to humidity, and is easy to hydrolyze and self-polymerize under humid environment. In addition, reactions involving silane coupling agents usually form a multilayer structure, resulting in the uncertainty of surface functionalized structure.

### SUMMARY OF THE INVENTION

The present disclosure aims to provide a novel biochip substrate, and proposes a preparation method and application thereof. The novel biochip substrate is based on silicon materials, with surface derivatized of vinyl sulfone groups featuring high-density carbon-carbon double bonds for immobilization of biomacromolecules, with a structural formula I:

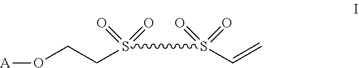

wherein, A is silicon-based substrate; the wavy bond (wavy line) in formula I represents link unit, selecting from alkyl chain, aryl chain, and polyethylene glycol chain.

In someone embodiments, the compound with vinyl sulfone groups at both ends is divinyl sulfone.

The biochip substrate can be used for immobilizing various biomolecules with mild immobilization conditions, simple operation and low-background; the preparation method thereof does not need complex pretreatment process, and has high operability and reproducibility, mild reaction conditions, simple operation, and is environmentally friendly, which is a kind of biochip substrate with great potential.

The novel biochip substrate mentioned above, is obtained by immersing silicon-based substrate in a solution of compound with vinyl sulfone groups at both ends, and reacting at 25-100° C. under the action of catalyst. The preparation method includes the following steps: dissolving a compound with structural formula II containing vinyl sulfone groups at both ends in an aprotic polar solvent, immersing the silicon-based substrate in the solution to react at 25-100° C. for 1-24 hours under the action of catalyst to prepare the biochip substrate.

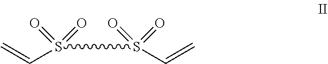

For the present preparation method of novel biochip substrate, as preferred, the surface of the silicon-based substrate material contains silicon hydroxyl groups.

For the present preparation method of novel biochip substrate, a preferred material of the silicon-based substrate is silicon wafer, glass, optical fiber or quartz wafer.

For the present preparation method of novel biochip substrate, a preferred catalyst is trisubstituted organic phosphine or trisubstituted organic amine.

For the present preparation method of novel biochip substrate, a preferred trisubstituted organic phosphine is triphenylphosphine, tri-i-sopropylphosphine, benzyldiphenylphosphine, or dimethylphenylphosphine.

For the present preparation method of novel biochip substrate, preferably, a dosage of the catalyst is 1-20% of the amount of the substance of the compound containing vinyl sulfone groups at both ends.

For the present preparation method of novel biochip substrate, a preferred aprotic polar solvent is selected from acetonitrile, acetone, N,N-dimethylformamide, dimethylsulfoxide, tetrahydrofuran, dioxane, dichloromethane, and chloroform. For the present preparation method of novel biochip substrate, a preferred reaction temperature is 25-60° C.

For the present preparation method of novel biochip substrate, a preferred reaction time is 4-8 hours.

The biochip substrate in the present disclosure has great potentials to be used as a broad-spectrum biochip substrate for a wide variety of applications, for example, functioning as protein chip, DNA chip and fluorescent chip.

### BENEFICIAL EFFECTS

Compared with existing biochip substrates, the biochip substrate in the present disclosure has high-density active double bonds which can be used for the immobilization of various biomolecules;

and the biochip substrate in the present disclosure has low-background, which is a biochip substrate with great potential. The preparation method in the present disclosure does not require complex pretreatment processes, and has mild immobilization conditions, and high operability and reproducibility, mild reaction conditions, simple operation, and is environmentally friendly.

### BRIEF DESCRIPTION OF THE DRAWINGS

FIG. 1 shows the changes of static water contact angles before and after the reaction of divinyl sulfone modified silicon wafer.

FIG. 2 shows the performances of the reaction of divinyl sulfone modified silicon wafer under different catalysts.

FIG. 3 shows the effects of different temperatures on the reaction of divinyl sulfone modified silicon wafer.

FIG. 4 shows a comparison of X-ray photoelectron spectra of a biochip substrate and that of a silicon-based substrate.

FIG. 5 shows a representation of static water contact angle between a biochip substrate and a silicon-based substrate.

FIG. 6 is a scan of fluorescence chip prepared by sulfo-cyanine3 amine immobilized by biochip substrates.

### DETAILED DESCRIPTION OF THE PREFERRED EMBODIMENTS

The embodiments are detailed descriptions of the content of the present disclosure without limiting the present disclosure in any way.

The surface of the novel biochip substrate in the present disclosure is vinyl sulfone groups, and the silicon-based material is used as the substrate, a compound containing vinyl sulfone groups at both ends is used for surface modification, the modified vinyl sulfone functional group can react with amino group and sulfydryl in the biomolecular to prepare the biochip.

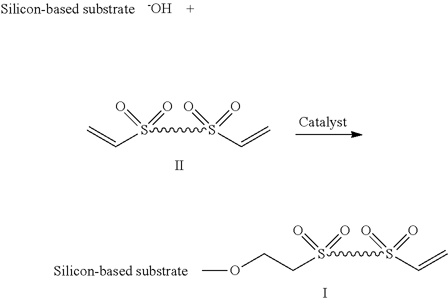

### Embodiment 1: Divinyl Sulfone (DVS) Functionalized Monocrystalline Silicon

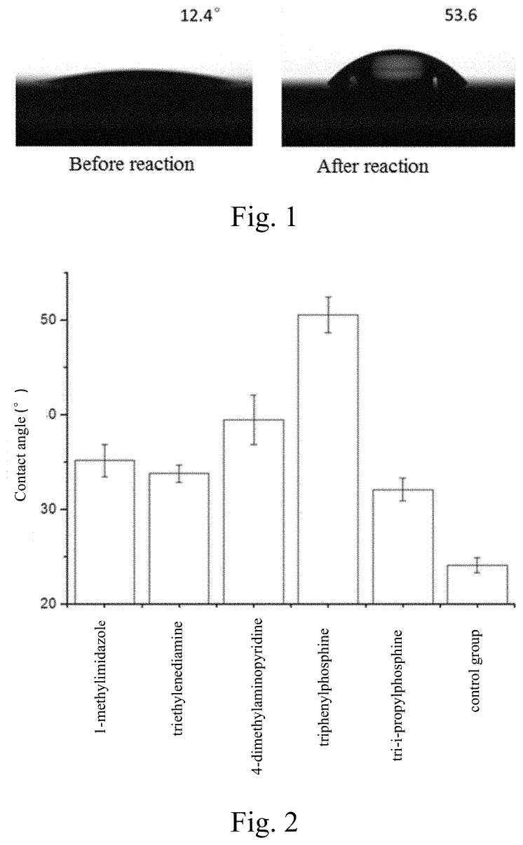

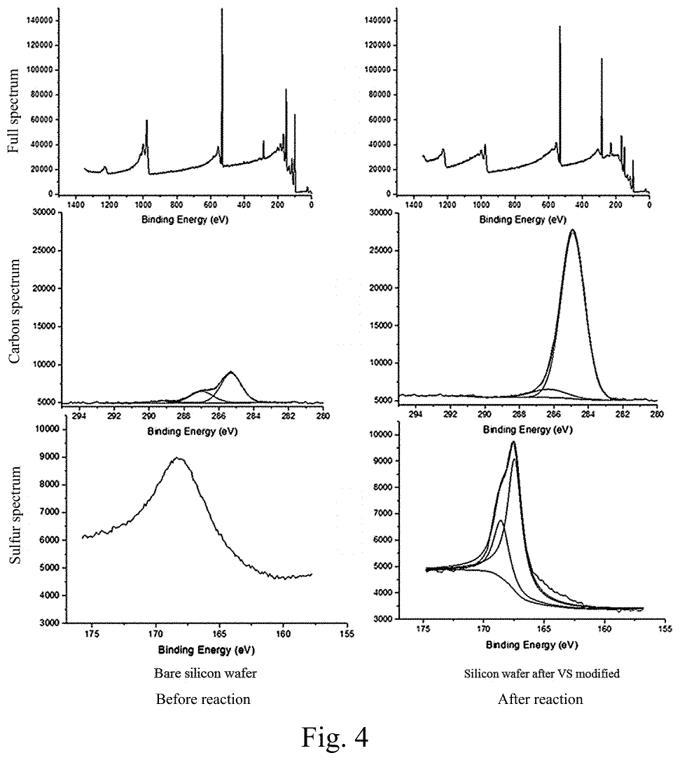

The silicon wafer was immersed in a “piranha” solution (concentrated sulfuric acid: H₂O₂ (30%) =3:1) at 90° C. for 2 hours to clean the surface, the cleaned silicon wafer was put into ultrapure water for ultrasonic cleaning for three times with five minutes each time, and the silicon wafer was immersed in a divinyl sulfone solution (500 mM) after nitrogen blow-dried, then reacted with acetonitrile as a solvent and catalyzed by triphenylphosphine (10 mM) at 60° C. for 6 hours. And then the silicon wafer was taken out and cleaned by ultrasonic in acetonitrile, then nitrogen blow-dried. The static water contact angles on the silicon wafer surface before and after the reaction were measured respectively, and the results were shown in FIG. 1. The surface of the cleaned silicon wafer was hydroxyl groups with better hydrophilicity, the static water contact angle was 12.4° (before reaction in FIG. 1); after modification by divinyl sulfone, the surface was vinyl sulfone groups with hydrophobicity increasing, and the static water contact angle increased to 53.6° (after reaction in FIG. 1). X-ray photoelectron spectroscopy was used to detect the silicon wafer before and after reaction, and the results were shown in FIG. 4. It can be seen from the carbon spectrum that the surface of the silicon wafer contains a small amount of carbon atoms before the reaction, which is due to the silicon wafer slightly polluted by air exposure after cleaning; the carbon atoms on the surface of silicon wafer increased significantly after reaction because divinyl sulfone contains carbon atoms. It can be seen from the sulfur spectrum in FIG. 4 that there is no S element on the surface of the silicon wafer, and the spectral peak was a loss peak caused by the presence of Si element; a characteristic spectral peak of sulfur element appeared after the reaction, and the position of the spectral peak was characteristic of sulfone group, which proved that the divinyl sulfone was successfully modified to the surface of silicon wafer. This conclusion is also supported by the element relative content on the surface of silicon wafer before and after the reaction obtained from

X-ray photoelectron spectroscopy (as shown in Table 1).

TABLE 1
Element relative content of the substrate
before and after reaction/Atmo %

| Element | Before reaction | After reaction |
|---|---|---|
| Si | 50.8 | 20.0 |
| C | 11.1 | 48.6 |
| O | 39.6 | 25.9 |
| S | ND | 4.8 |
| N | 0.5 | 0.7 |

### Embodiment 2: Divinyl sulfone (DVS) Functionalized Monocrystalline Silicon with Different Catalysts

1-methylimidazole, triethylenediamine, 4-dimethylaminopyridine, triphenylphosphine, and tri-i-propylphosphine were respectively used as catalysts (no catalyst was used in the control group), and other experimental processes and experimental conditions were the same as embodiment 1. The static water contact angle of the silicon wafer after reaction was measured, and the results were shown in FIG. 2. Compared with the control group, the contact angles all increased under the conditions with five catalysts, and the maximum increase was catalyzed by triphenylphosphine, indicating that the five catalysts have catalytic effect on the reaction, among which triphenylphosphine has the greatest effect.

### Embodiment 3: Divinyl Sulfone (DVS) Functionalized Monocrystalline Silicon with Different Temperatures

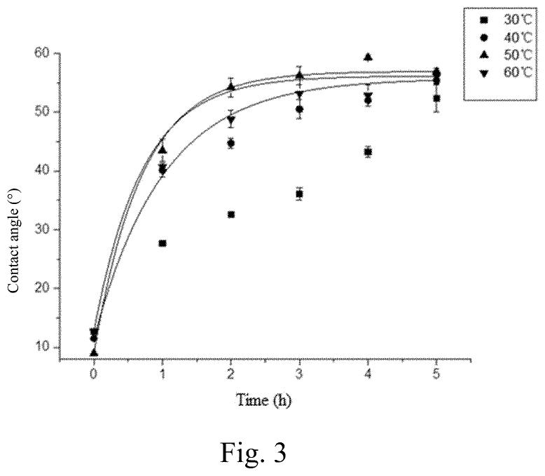

30° C., 40° C., 50° C. and 60° C. were respectively used as reaction temperature, and other experimental processes and experimental conditions were the same as embodiment 1. The static water contact angle of the silicon wafer surface was measured each 1 hour, and the results were shown as FIG. 3. Under the four reaction temperatures, the static water contact angle increased with the increase of the reaction time, indicating that the reaction can be carried out at all four temperatures; and the higher the temperature, the faster the static water contact angle increased, indicating that the increase of temperature accelerates the reaction rate.

### Embodiment 4: Preparation of Vinyl Sulfone Substrate Using Optical Grade Slide as Substrate

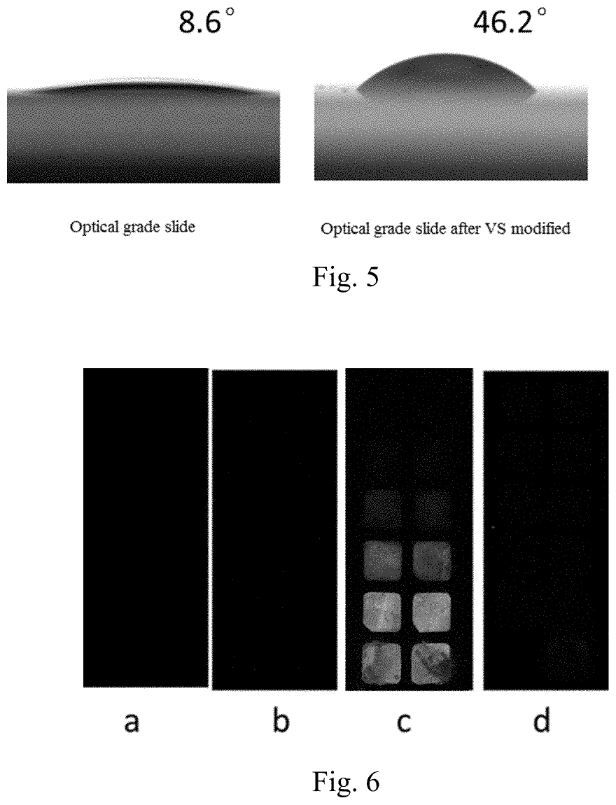

An optical grade slide was immersed in a divinyl sulfone solution (500 mM) and reacted with acetonitrile as a solvent at 60° C. for 6 hours under the catalysis of triphenylphosphine. After that, the optical grade slide was taken out and cleaned by ultrasonic in acetonitrile, then nitrogen blow-dried. The static water contact angles of the silicon wafer before and after the reaction were measured respectively, and the results were shown in FIG. 5. The surface of the slide before reaction was hydroxyl group with good hydrophilicity, and the static water contact angle was 8.6°; after modification by divinyl sulfone, the surface of the slide was vinyl sulfone group with hydrophobicity increasing, and the static water contact angle increased to 46.2°.

### Embodiment 5: Immobilizing Sulfo-Cyanine3 Amine by Vinyl Sulfone Substrate

Sulfo-cyanine3 amine is a kind of water-soluble fluorescent dye with amino group, with a structural formula III

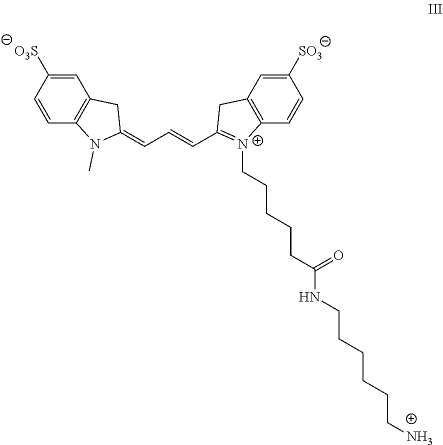

Sulfo-cyanine3 amine was dissolved in HEPES buffer (pH=9, 50 mM) to prepare the reaction liquid with concentration of 0.00001 mg/ml, 0.0001 mg/ml, 0.001 mg/ml, 0.01 mg/ml, 0.1 mg/ml and 1 mg/ml respectively. Multi-sample independent reaction fence was pasted on the vinyl sulfone substrate in embodiment 4, then the coverglass was applied, and the reaction liquid was added in independent reaction chamber at different time from the sample holes, each reaction solution was added in two independent reaction chambers to react for 6 hours at 25° C. under wet box condition. After reaction, removing the coverglass, the reaction liquid was taken out, and the fence was removed, then the substrate was put into ultra-pure water for ultrasonic cleaning for 10 min and nitrogen blow-dried. The substrate was scanned by crystal core TMLUXScan-10K/B (CapitalBio Corportation) with the scanning parameters setting of Laser/PMT=1/600. The results were shown as FIG. 6, wherein FIG. 6a is optical grade slide; FIG. 6b is vinyl sulfone substrate; FIG. 6c is the substrate added different reaction liquid and the concentration increased from top to bottom; FIG. 6d is optical grade slide treated the same way as that shown in FIG. 6c. No fluorescence was observed in FIG. 6a of optical glass slide and FIG. 6b of vinyl sulfone substrate, indicating that the fluorescence background of vinyl sulfone substrates is very low, which would not affect the application of biochip. In FIG. 6c, obvious fluorescence could be observed, and the fluorescence intensity increased with the increase of the concentration of the reaction solution; while in FIG. 6d no fluorescence was observed, indicating that sulfo-cyanine3 amine was successfully immobilized on the surface of the vinyl sulfone substrate, and the vinyl sulfone substrate could be used for immobilizing molecules with amino groups.

For those skilled in the art, without departing from the scope of technical solutions of the present disclosure, many possible variations and modifications can be made to the technical solutions of the present disclosure by using the technical contents disclosed above, or to modify the equivalent embodiments with equivalent changes. Therefore, any simple changes, equivalent changes and modifications of the above embodiments made according to the technical essence of the present disclosure without departing from the technical solutions of the present disclosure shall belong to the scope of protection of the present disclosure.

1. A novel biochip substrate, wherein a surface of the novel biochip substrate contains vinyl sulfone groups with a structural formula I: 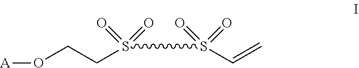
wherein, A is silicon-based substrate.

2. A preparation method of the biochip substrate according to claim 1, comprising the following steps: dissolving a compound with structural formula II containing vinyl sulfone groups at both ends in an aprotic polar solvent, and immersing the silicon-based substrate in the solution to react at 25-100° C. for 1-24 hours under an action of catalyst to prepare the biochip substrate

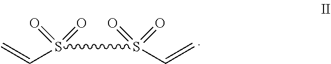

3. The preparation method of the biochip substrate according to claim 2, wherein the surface of the silicon-based substrate material contains silanol groups.

4. The preparation method of the substrate according to claim 3, wherein the silicon-based substrate is silicon wafer, glass, optical fiber or quartz wafer.

5. The preparation method of the biochip substrate according to claim 2, wherein the catalyst is trisubstituted organic phosphine or trisubstituted organic amine.

6. The preparation method of the biochip substrate according to claim 5, wherein the trisubstituted organic phosphine is triphenylphosphine, tri-i-sopropylphosphine, benzyldiphenylphosphine or dimethylphenylphosphine.

7. The preparation method of the biochip substrate according to claim 2, wherein a dosage of the catalyst is 1-20% of the amount of the substance of the compound containing vinyl sulfone groups at both ends.

8. The preparation method of the biochip substrate according to claim 2, wherein the aprotic polar solvent is acetonitrile, acetone, N,N-dimethylformamide, dimethyl sulfoxide, tetrahydrofuran, dioxane, dichloromethane or chloroform.

9. The preparation method of the biochip substrate according to claim 2, wherein the reaction temperature is 25-60° C.

10. The preparation method of the biochip substrate according to claim 2, wherein the reaction time is 4-8 hours.

11. Applications of the biochip substrate in the field of biochip according to claim 1, comprising applications in the field of protein chip, DNA chip and fluorescent chip.
